(19) United States
(12) Patent Application Publication
(10) Pub. No.: US 2025/0101475 A1
(43) Pub. Date: Mar. 27, 2025

(54) MONOOXYGENASE MUTANTS FOR BIOSYNTHESIS OF 2,6-BIS(HYDROXYMETHYL)PYRIDINE AND A METHOD FOR PREPARATION OF 2,6-BIS(HYDROXYMETHYL)PYRIDINE USING THE SAID MONOOXYGENASE MUTANTS

(71) Applicant: VIO Chemicals AG, Zurich (CH)

(72) Inventors: Sven Panke, Basel (CH); Martin Held, Basel (CH); Tsvetan Kardashliev, Basel (CH)

(21) Appl. No.: 18/725,189

(22) PCT Filed: Dec. 29, 2022
(86) PCT No.: PCT/EP2022/088056

§ 371 Date: Jun. 28, 2024

(30) Foreign Application Priority Data
Dec. 29, 2021 (WO) PCT/EP2021/087820

(51) Int. Cl. C12P 17/12; C12N 9/02; C12N 9/04; C12N 9/10; C12N 15/52; C12N 15/70; C12P 17/18

(52) U.S. Cl. CPC C12P 17/12; C12N 9/0006; C12N 9/0077; C12N 9/1029; C12N 15/52; C12N 15/70; C12P 17/182; C12Y 101/01001; C12Y 101/0109; C12Y 114/15; C12Y 203/01

(57) ABSTRACT

The present invention relates to the provision of an enzymatic method for the preparation of 2,6-bis(hydroxy methyl)pyridine starting from 2,6-lutidine using a mutated xylene monooxygenase enzyme, termed ppXMO, comprising a xylM subunit and a xylA subunit from Pseudomonas putida, wherein said mutated enzymes harbor an amino acid exchange at position 116 of the amino acid sequence of XylM component. The essence of the invention is that the methionine (M) at this position is replaced with an aminoacid selected in the group consisting of asparagine (N), lysine (K), arginine (R) and glycine (G), which surprisingly results in a direct methyl hydroxylation of 6-methyl-2-pyridine methanol resulting in improved overall process yield, less side products are produced, avoidance of toxic reaction intermediates and minimizing the need for involvement of endogenous reductase enzymes as well as NADPH and its regeneration. Other enzymes related to XylM of P. putida harbouring the same amino acid exchange at the highly conserved region around position 116 or its equivalent also exhibit similar improved characteristics.

### TECHNICAL FIELD OF THE INVENTION

The present invention belongs to the field of biochemistry, more precisely to enzymes for different applications and genetic engineering for preparation of mutated enzymes. The invention also belongs to the field of organic chemistry. The invention relates to monooxygenase mutants for biosynthesis of 2,6-bis(hydroxymethyl)pyridine (Formula I) and a method for preparation of 2,6-bis(hydroxymethyl)pyridine using said monooxygenase mutants.

### BACKGROUND OF THE INVENTION

2,6-bis(hydroxymethyl)pyridine (Formula I) is a compound which can serve as a versatile intermediate in the preparation of other complex products. The hydroxyl group can be converted to other functional groups such as aldehyde groups, halogenated hydrocarbons, amino groups etc., which are then used in the preparation of further useful compounds. Furthermore, owing to the substitution on positions 2 and 6, 2,6-bis(hydroxymethyl)pyridine can also be used in the synthesis of macrocyclic compounds. One example is pyclen, an azamacrocyclic framework, which incorporates an aromatic pyridine moiety to the 12-membered macrocyclic unit.

The compound of formula I can be synthesized from 2,6-lutidine II, which is an easily accessible starting material, by oxidation with KMnO₄ toward the respective dicarboxylic acid, conversion to the respective ester and finally reduction of the ester groups to alcohols (Journal of Dispersion Science and Technology 2006, 27, p. 15-21). The cited reference is silent with respect to the yield of this three-step conversion. Additionally, this synthetic approach is tedious, as it requires three overall steps and several intermediate isolations accompanied by purifications.

Patent application CN105646334A disclosed the above synthetic approach by eliminating the ester conversion step, i.e. the dicarboxylic acid is first isolated and then directly converted to the bis-alcohol. The Chinese patent application reports a combined yield of 64% for this two-step process, which is a moderate yield for such a short synthesis.

Egorov et al reported in 1985 (Prikladnaya Biokhimiya i Mikrobiologiya, 21 (3), pp. 349-353) that suspensions of certain non-multiplying cells were found able to hydroxylate 2,6-dimethylpyridine to 2-methyl-6-hydroxymethylpyridine. A small quantity of 2,6-bis(hydroxymethyl)pyridine was found to be formed only by the species Sporotrichum sulfurescens ATCC 7159. It was suggested that the polarity of the substrate is increased by insertion of the first hydroxyl group, which hinders the oxidation of the second methyl group. The document disclosed that the yield could not be substantially increased by increasing the duration of the transformation reaction.

It would be desirable to develop a selective method to produce 2,6-bis(hydroxymethyl)pyridine (Formula I) from 2,6-lutidine (Formula II) without the need to isolate intermediates and with high yield, which is cost-effective from the prospect of industrial manufacturing and also more sustainable.

Furthermore, it is the aim of the invention to provide enzyme variants that would improve the efficiency of production of 2,6-bis(hydroxymethyl)pyridine from 2,6-lutidine.

### STATE OF THE ART

A recently filed patent application PCT/EP2021/068920, which at the time of drafting has not been published, discloses a process for the transformation of 2,6-lutidine II to 2,6-bis(hydroxymethyl)pyridine I,

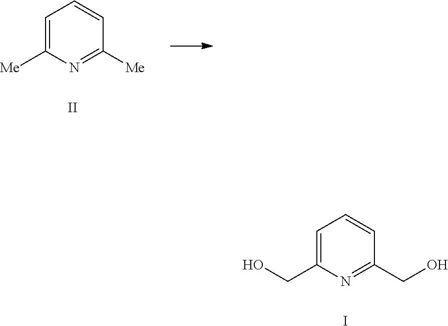

wherein the transformation is performed in the presence of enzymes and wherein the transformation may proceed via the formation of 6-methyl-2-hydroxypyridine (Formula III).

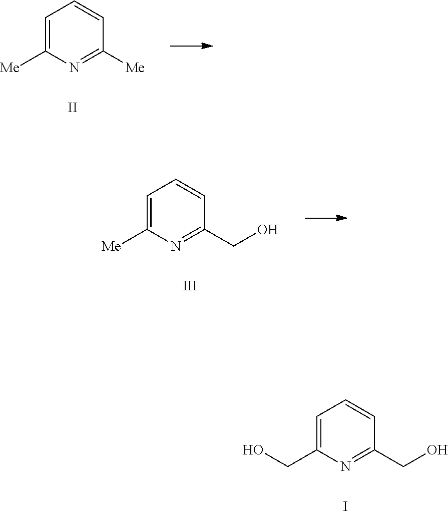

The enzymes used in the process are oxidoreductases, preferably NAD (P) H dependent oxidoreductases, which use molecular oxygen to oxidize 2,6-lutidine II. A possible oxidoreductase enzyme according to this document is:
a xylene monooxygenase enzyme encoded by the xylM and xylA genes of Pseudomonas putida (Arthrobacter siderocapsulatus), termed ppXMO or
a XylMA-like enzyme of:
   Alteromonas macleodii or
   Tepidiphilus succinatimandens or
   Novosphingobium kunmingense, or
   Hyphomonas oceanitis or
   Sphingobium sp. 32-64-5 or
   Halioxenophilus aromaticivorans; or
a XylMA-like enzyme with more than 70% sequence identity on the amino acid level.

Albeit the process according to this solution is cost-effective from the prospect of industrial production, the above-mentioned reaction pathway is suboptimal for several reasons including:
highly toxic nature of the formed intermediates, 6-methyl-2-pyridinecarbaldehyde and 6-(hydroxylmethyl)pyridine-2-carbaldehyde,
formation of 6-methyl-2-pyridinecarboxylic acid as a side product,
involvement of at least one endogenous enzyme, and
high demand for both NADH and NADPH cofactors.

These factors negatively influence the overall process yield. Thus, the technical problem is to address these disadvantages.

### SUMMARY OF THE INVENTION

The present invention discloses an enzymatic method for the preparation of compound of formula I, starting from 2,6-lutidine (formula II). The method disclosed herein comprises one step, said step comprising the presence of an enzyme, which can perform the double oxidation in a selective manner.

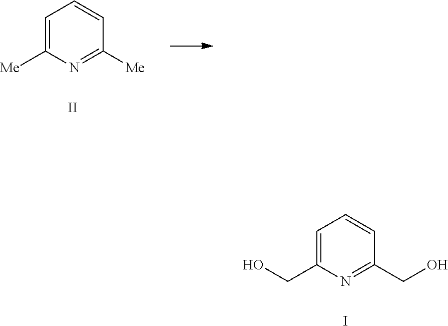

With the aim to address the disadvantages of the method using a wild-type xylene monooxygenase enzyme, ppXMO, comprising a XylM subunit and a XylA subunit from Pseudomonas putida (Arthrobacter siderocapsulatus), mutated enzymes have been generated using genetic engineering, wherein said mutated enzymes harbor an amino acid exchange at position 116 of the amino acid sequence of XylM. The essence of the invention is that the methionine (M) at the position 116 in a highly conserved region is replaced with any different amino acid, preferably with an amino acid selected in the group consisting of asparagine (N), lysine (K), arginine (R) and glycine (G), which surprisingly results in a direct methyl hydroxylation of 6-methyl-2-pyridine methanol (Formula III). The mutant enzymes have been observed to:
improve overall process yield by at least 50%,
improve final product profile, meaning fewer side products are produced,
facilitate the process control during preparative synthesis (in a bioreactor),
alleviate the toxicity of reaction intermediates,
minimize/eliminate the need for involvement of endogenous reductase enzymes,
minimize/eliminate the need for NADPH and its regeneration.

Other enzymes related to XylM component of P. putida (A. siderocapsulatus) harbouring the same amino acid exchange within the highly conserved region around position 116 also exhibit similar improved characteristics and are thus also suitable for use in the preparation of compound of formula I, starting from 2,6-lutidine. In some XylM related enzymes, such as XylM from Halioxenophilus aromaticivorans (GenBank: BBB44451.1), the equivalent position of the methionine is 134, while in mutant ntnMO derived from the ntnMO WT (GenBank: AAC38359.1) the equivalent position is 116 and instead of methionine tryptophan (W) is present. Homology of 50% on the amino acid level to the XylM component of P. putida is required to ensure retained enzymatic activity and the effect of described mutation.

The bis-OH product of the method according to the invention may be used to prepare the respective bis-LG compound, most commonly 2,6-Bis(chloromethyl)pyridine, 2,6-Bis(bromomethyl)pyridine, 2,6-Bis(mesyloxymethyl)pyridine and 2,6-Bis(tosyloxymethyl)pyridine.

In addition, the above-mentioned method and used enzymes may be used in methods for preparation of other compounds, diagnostic complexes, or other products. In a possible embodiment, the method according to the invention may be a step in a method for preparation of pyridine-based tetra-aza heterocycles, preferably pyclen (3,6,9,15-tetraazabicyclo[9.3.1]pentadeca-1(15),11,13-triene). Also, products of the method according to the invention may be used in methods for preparation of pyridine-based tetra-aza heterocycles, preferably pyclen.

Furthermore, the method according to the invention and/or the method for preparation of pyridine-based tetra-aza heterocycles, preferably pyclen, and/or products resulting therefrom may be used in methods for preparation of various diagnostic complexes or other compounds. In a most preferred embodiment, the method according to the invention or its products are used in preparation of gadopiclenol (C₃₅H₅₄GdN₇O₁₅, INN, trade name Elucirem), which is a contrast agent used with magnetic resonance imaging (MRI) to detect and visualize lesions with abnormal vascularity in the central nervous system and in the body.

### Definitions

The following terms shall have, for the purposes of this application, including the claims appended hereto, the respective meanings set forth below. It should be understood that when reference herein is made to a general term, such as enzyme, solvent, etc. one skilled in the field may make appropriate selections for such reagents from those given in the definitions below, as well as from additional reagents recited in the specification that follows, or from those found in literature references in the field.

The term “enzymatic process” or “enzymatic method” as used herein denotes a process or method employing an enzyme or microorganism.

The term “microbial cell” refers to wild type microbial cell, wild type microbial cell or genetically modified unicellular microorganism, also called recombinant, that serves as a host for production of functional entities (enzymes) participating in the enzymatic process. The terms host and cell are used interchangeably throughout the present invention.

The term “recombinant cell” denotes that the microbial cell further harbours heterologous DNA encoding enzyme functionality supplied in the form of genomic integration or plasmid DNA.

The term “feeding rate” denotes the quantity of substance (e.g., glucose, glycerol, lutidine, or any other nutrient, cofactor or similar) per unit of time and volume added to the reaction medium within the course of the enzymatic process.

The term “reaction medium” refers to any growth medium used to perform a process which comprises enzymes. The said medium is able to carry the starting material, the enzyme either alone or as part of a cell and the product and byproducts. Usually, the reaction medium is an aqueous solvent.

The term “cofactor regeneration system” denotes an enzyme or a set of enzymes that reduce a biological cofactor, preferably NAD+ to NADH, NADP+ to NADPH, more preferably of NAD+ to NADH using biocompatible substrates such as standard carbon sources (glucose, glycerol), an alcohol or an organic acid (e.g. formate).

The term “formate” refers to the anion generated by the respective salts, e.g., sodium formate.

The term “nutrient” means organic or inorganic molecules that can serve as sources of carbon (e.g., glucose, glycerol), nitrogen (e.g., ammonia, amino acids), phosphorus (phosphate salts, phytate), micronutrients (metal ions, vitamins).

The enzymes employed in the present invention are derived from microbial genomes. The genes may be codon optimized and synthetically prepared or isolated from the respective host (e.g. by PCR). For example, they may be cloned (e.g., using restriction enzymes and DNA ligase) in suitable expression vectors or integrated on the genome of the recombinant host to yield genetically engineered host cells.

Additionally, it should be understood in the methods of preparation and claims herein, that the pronoun “a”, when used to refer to a reagent, such as “a base”, “a solvent” and so forth, is intended to mean “at least one” and thus, include, where suitable, single reagents as well as mixtures of reagents.

### DETAILED DESCRIPTION OF THE INVENTION

The present invention discloses an enzymatic method for the preparation of compound of 2,6-bis(hydroxymethyl)pyridine (Formula I). It is possible to obtain compound of formula I starting from readily available formula II in the presence of enzymes in high yields and without formation of significant amounts of by-products (side products) using a wild type ppXMO from P. putida (A. siderocapsulatus) having the following amino acid sequence SEQ ID NO 1.

SEQ ID NO 1:
MDTLRYYLIPVVTACGLIGFYYGGYWVWLGAATFPALMVLDVILPKDFS
ARKVSPFFADLTQYLQLPLMIGLYGLLVFGVENGRIELSEPLQVAGCIL
SLAWLSGVPTLPVSHELMHRRHWLPRKMAQLLAMFYGDPNRDIAHVNTH
HLYLDTPLDSDTPYRGQTIYSFVISATVGSVKDAIKIEAETLRRKGQSP
WNLSNKTYQYVALLLALPGLVSYLGGPALGLVTIASMIIAKGIVEGFNY
FQHYGLVRDLDQPILLHHAWNHMGTIVRPLGCEITNHINHHIDGYTRFY
ELRPEKEAPQMPSLFVCFLLGLIPPLWFALIAKPKLRDWDQRYATPGER
ELAMAANKKAGWPLWCESELGRVASI

The inventors have surprisingly found that mutated ppXMO enzyme from P. putida (A. siderocapsulatus), prepared by genetic engineering, harbouring an amino acid exchange at position 116 of the amino acid sequence of XylM component, enables direct methyl hydroxylation of 6-methyl-2-pyridine methanol III. The methionine (M) at position 116 in the wild-type XylM component is replaced with any amino acid different from M, preferably with an amino acid selected in the group consisting of asparagine (N), lysine (K), arginine (R) and glycine (G), wherein the preferred choice is glycine (G). The invention thus relates to the mutated enzymes as well as to all polynucleic acids encoding the mutated enzymes having the following amino acid sequences:

SEQ ID NO 2:
MDTLRYYLIPVVTACGLIGFYYGGYWVWLGAATFPALMVLDVILPKDFS
ARKVSPFFADLTQYLQLPLMIGLYGLLVFGVENGRIELSEPLQVAGCIL
SLAWLSGVPTLPVSHELGHRRHWLPRKMAQLLAMFYGDPNRDIAHVNTH
HLYLDTPLDSDTPYRGQTIYSFVISATVGSVKDAIKIEAETLRRKGQSP
WNLSNKTYQYVALLLALPGLVSYLGGPALGLVTIASMIIAKGIVEGFNY
FQHYGLVRDLDQPILLHHAWNHMGTIVRPLGCEITNHINHHIDGYTRFY
ELRPEKEAPQMPSLFVCFLLGLIPPLWFALIAKPKLRDWDQRYATPGER
ELAMAANKKAGWPLWCESELGRVASI
SEQ ID NO 3:
MDTLRYYLIPVVTACGLIGFYYGGYWVWLGAATFPALMVLDVILPKDFS
ARKVSPFFADLTQYLQLPLMIGLYGLLVFGVENGRIELSEPLQVAGCIL
SLAWLSGVPTLPVSHELNHRRHWLPRKMAQLLAMFYGDPNRDIAHVNTH
HLYLDTPLDSDTPYRGQTIYSFVISATVGSVKDAIKIEAETLRRKGQSP
WNLSNKTYQYVALLLALPGLVSYLGGPALGLVTIASMIIAKGIVEGFNY
FQHYGLVRDLDQPILLHHAWNHMGTIVRPLGCEITNHINHHIDGYTRFY
ELRPEKEAPQMPSLFVCFLLGLIPPLWFALIAKPKLRDWDQRYATPGER
ELAMAANKKAGWPLWCESELGRVASI
SEQ ID NO 4:
MDTLRYYLIPVVTACGLIGFYYGGYWVWLGAATFPALMVLDVILPKDFS
ARKVSPFFADLTQYLQLPLMIGLYGLLVFGVENGRIELSEPLQVAGCIL
SLAWLSGVPTLPVSHELKHRRHWLPRKMAQLLAMFYGDPNRDIAHVNTH
HLYLDTPLDSDTPYRGQTIYSFVISATVGSVKDAIKIEAETLRRKGQSP
WNLSNKTYQYVALLLALPGLVSYLGGPALGLVTIASMIIAKGIVEGFNY
FQHYGLVRDLDQPILLHHAWNHMGTIVRPLGCEITNHINHHIDGYTRFY
ELRPEKEAPQMPSLFVCFLLGLIPPLWFALIAKPKLRDWDQRYATPGER
ELAMAANKKAGWPLWCESELGRVASI
SEQ ID NO 5:
MDTLRYYLIPVVTACGLIGFYYGGYWVWLGAATFPALMVLDVILPKDFS
ARKVSPFFADLTQYLQLPLMIGLYGLLVFGVENGRIELSEPLQVAGCIL
SLAWLSGVPTLPVSHELRHRRHWLPRKMAQLLAMFYGDPNRDIAHVNTH
HLYLDTPLDSDTPYRGQTIYSFVISATVGSVKDAIKIEAETLRRKGQSP
WNLSNKTYQYVALLLALPGLVSYLGGPALGLVTIASMIIAKGIVEGFNY
FQHYGLVRDLDQPILLHHAWNHMGTIVRPLGCEITNHINHHIDGYTRFY
ELRPEKEAPQMPSLFVCFLLGLIPPLWFALIAKPKLRDWDQRYATPGER
ELAMAANKKAGWPLWCESELGRVASI

The underlined parts of above-given sequences indicate a highly conserved region, wherein replacement of M at position 116 causes a significant change in enzyme properties compared to the WT. In some ppXMO related enzymes, such as hoXMO, the equivalent position of the methionine is 134, while in ntnMO the equivalent position is 116 and instead of methionine tryptophan (W) is present. The effect of mutations is the same or highly similar. The mutant enzymes have been observed to:
improve overall preparative synthesis process yield (especially in a bioreactor), wherein usually the yield is improved for at least 50%,
improve final product profile, meaning that none or fewer side products are produced,
facilitate the process control during preparative synthesis (in a bioreactor),
alleviate the issues with toxicity of reaction intermediates, which is a direct consequence of the mutated enzyme ability to convert compound of formula I and III simultaneously (as opposed to WT that must first deplete compound II before starting to convert compound III),
minimize the need for involvement of endogenous enzymes,
minimize/eliminate the need for NADPH and its regeneration.

Less preferred mutations at position 116 as well as the preferred mutations may be further improved if combined with other mutations of XylMA.

Other enzymes related to ppXMO of P. putida (A. siderocapsulatus) harbouring the same amino acid exchange of methionine in the highly conserved region (equivalent to position 116 in XylM component) also exhibit similar improved characteristics and would be also suitable for use in the preparation of compound of formula I, starting from 2,6-lutidine. More preferably, the oxidoreductase enzyme is a xylene monooxygenase enzyme encoded by the xylM and xylA genes of P. putida (A. siderocapsulatus), or a XylMA-like enzyme of Alteromonas macleodii or of Tepidiphilus succinatimandens or of Novosphingobium kunmingense or of Hyphomonas oceanitis or of Sphingobium sp. 32-64-5 or of Halioxenophilus aromaticivorans or a XylM-like enzyme with more than 50% sequence identity on the amino acid level of the entire XylM sequence. Even more preferably, the oxidoreductase enzyme is a xylene monooxygenase enzyme, ppXMO, encoded by the xylM and xylA genes of Pseudomonas putida (Arthrobacter siderocapsulatus). Alternatively, the preferred option is also a XylMA-like enzyme from H. aromaticivorans having an amino acid sequence SEQ ID NO: 6, which is:

SEQ ID NO 6:
MDTIRYYLIPLVSACGALGFYYGGDWVWLGAATFPSLMILDVLLPRDYE
ERKVSPFFADLTQYLQLPLMIAMYGFLIFGVREGRIDLGEPVQFLGSIL
SLAWLSGVPTLPVSHELMHRRHWLPRRMAQLLATFYGDPNRDIAHVNTH
HLELDTPLDSDTPFRGQTMYSFVVSATVGSVMDAAKIEAETLRRKGKSP
WHLSNKMYQYVMLLIALPGVVTYFGGAESGLVTIISMLIAKAIVEGFNY
FQHYGLVREIGHPILLHHAWNHMGMIVRPLGCEITNHINHHLDGYTRFY
YLHPEKEAPQMPSLFLCFLLGLVPPLWENLVAKPKLKDWDLQYATPGER
KLAMEANKNAGWPQWIPEAA

The process for the transformation of 2,6-lutidine II to 2,6-bis(hydroxymethyl)pyridine I

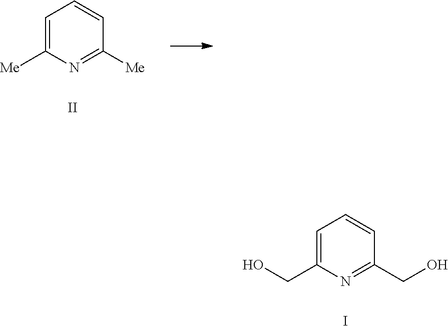

is performed using the aforementioned mutated enzymes. The disclosed enzyme may be used in the disclosed method according to techniques well known to the skilled person. They may be used as part of the cells producing them (whole cell catalysis) or in vitro, where the enzyme is available and is employed in the reaction media under appropriate reaction conditions.

In a preferred embodiment, the enzyme according to the invention is expressed in a microbial host. The microbial host may then be referred to as a recombinant microbial host. The recombinant host may further be tailored by genetic engineering. Preferable microbial hosts are Escherichia coli, Corynebacterium glutamicum, Bacillus subtilis, Pseudomonas putida, Rhodobacter sphaeroides, Streptomyces spp, Propionibacterium shermanii, Ketogulonigenium vulgare, Acinetobacter baylyi, Halomonas bluephagenesis. More preferable is Escherichia coli.

The skilled person is familiar with techniques for expressing certain enzymes in microbial hosts. Such techniques are exemplified in relevant textbooks, such as “Methods in Enzymology” (Book series, Elsevier, ISSN 0076-6879) or “Molecular Cloning” (ISBN 978-1-936113-42-2).

When the wild type enzyme is used, the enzymatic process at least partly proceeds via the formation of 6-methyl-2-hydroxypyridine III. In addition to compound of formula III, the enzymatic transformation of compound of formula II to compound of formula I proceeds via the formation of compound of formula IV, when the enzyme is a xylene monooxygenase enzyme.

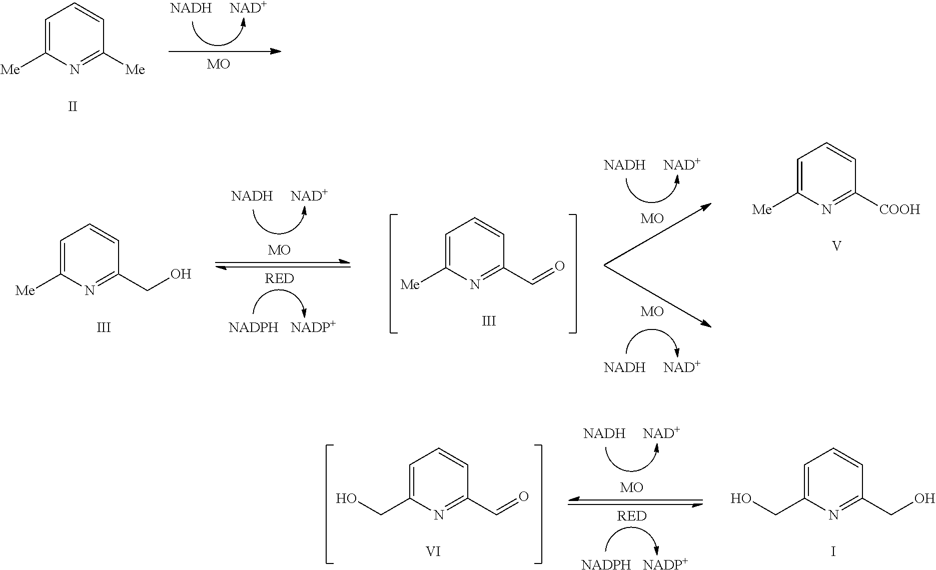

It is beneficial if 2,6-lutidine II is kept at a feeding rate suitable for maintaining a balance between the various transformations occurring within the enzymatic process. The feeding rate need not be constant, as long as it is adjusted according to the below embodiments. The feeding rate should also be at an appropriate level so as not to reach growth-inhibitory levels of 2,6-lutidine II.

In a preferred embodiment, the feeding rate of 2,6-lutidine II in the reaction medium is adjusted such that the concentration of 2,6-lutidine II does not exceed the value of 1 g/L, preferably 0.1 g/L, and more preferably 0.02 g/L in the reaction medium.

In another preferred embodiment, the feeding rate of 2,6-lutidine II in the reaction medium is adjusted such that the concentration of 2,6-lutidine II does not fall below the value of 10 mg/L, preferably 0.1 mg/L, more preferably 0.01 mg/L.

The method of the present invention is carried out in an aqueous medium. The aqueous medium is water, or deionized water, which may further comprise a buffering agents and nutrients.

The weight of biomass employed in the present process may be adjusted according to the skilled person's general knowledge.

The reaction medium temperature may be such that the enzyme retains its enzymatic activity. It is preferably maintained between 25 and 37° C., most preferably between 28 and 32° C.

The pH may be such that the enzyme and the recombinant strain retain their enzymatic activity. Preferably, the pH is between 6.0 and 8.0, more preferably 6.5-7.5 and even more preferably 7.0±0.1.

The dissolved oxygen tension (DOT) should be maintained above 0%. DOT drops as recombinant cells grow and biomass accumulates in the bioreactor and furthermore significantly once the substrate 2,6-lutidine II is added. It is therefore important that it is maintained above 0% or better above 3-5% in order for the biocatalytic reaction to take place. DOT can be controlled by energy input employed for mixing of the aqueous medium speed, the rate at which the bioreactor is aerated or supplementation of the supplied air with oxygen.

The rate of carbon-source, such as glucose or glycerol, feed may be adjusted as per skilled person's general knowledge.

The reaction time can be varied depending upon the amount of enzyme and its specific activity. It may further be adjusted by the temperature or other conditions of the enzymatic reactions, which the skilled person is familiar with. Typical reaction times are ranging between 1 hour and 72 hours.

In another embodiment, the process for the transformation of 2,6-lutidine II to 2,6-bis(hydroxymethyl)pyridine I, optionally involves presence of a dehydrogenase. The transformation may be performed directly in the microbial cell with no further engineering of the housekeeping dehydrogenases. In yet another embodiment, the microbial cell further synthesizes a dehydrogenase from another microbial cell. In even yet another embodiment, one or more housekeeping dehydrogenases are deactivated or engineered.

In a preferred embodiment, the microbial cell further synthesizes a dehydrogenase from another microbial cell and one or more housekeeping dehydrogenases are deactivated or engineered.

The enzyme which catalyses the oxidative transformation of the methyl groups of 2,6-lutidine II to the respective hydroxymethyl groups of 2,6-bis(hydroxymethyl)pyridine I and is employed in this embodiment is according to the previous embodiments.

As long as the skilled person arrives at a specific combination of enzymes, the expression of them within the same microbial host is a technique well known to the skilled person. Reference books have been provided above.

In a preferred embodiment, the dehydrogenase is NAD (P) H dependent or NADH dependent and preferentially NADH dependent.

In another preferred embodiment, the dehydrogenase catalyses the reduction of 6-methylpyridine-2-carboxaldehyde IV to 6-methyl-2-hydroxypyridine Ill or the reduction of 6-(hydroxymethyl)-2-pyridinecarbaldehyde V to 2,6-bis(hydroxymethyl)pyridine I. Preferably, the dehydrogenase catalyses both the reduction of 6-methylpyridine-2-carboxaldehyde IV to 6-methyl-2-hydroxypyridine III and the reduction of 6-(hydroxymethyl)-2-pyridinecarbaldehyde V to 2,6-bis(hydroxymethyl)pyridine I.

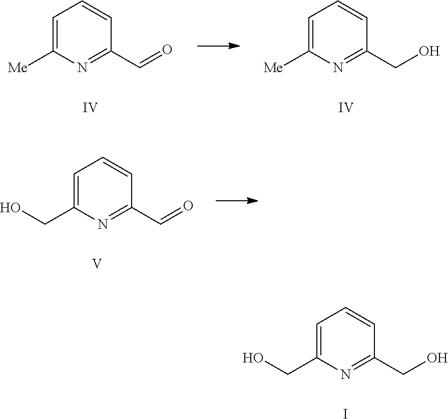

In another preferred embodiment, the dehydrogenase is selected from the list of the AKR from Kluyveromyces lactis, XylB from Acinetobacter baylyi ADP1, and AFPDH from Candida maris.

In another embodiment, there is provided a process for the transformation of 2,6-lutidine II to 2,6-bis(hydroxymethyl)pyridine I, wherein the transformation is performed in the presence of enzymes, which catalyse the oxidative transformation of the methyl groups of 2,6-lutidine II to the respective hydroxymethyl groups of 2,6-bis(hydroxymethyl)pyridine I, and additionally the presence of a co-factor regeneration system.

The enzyme, which catalyses the oxidative transformation of the methyl groups of 2,6-lutidine II to the respective hydroxymethyl groups of 2,6-bis(hydroxymethyl)pyridine I and is employed in this embodiment, is according to the previous embodiments.

The transformation may be performed directly in the microbial cell with no further engineering of the housekeeping dehydrogenases, as disclosed in previous embodiments.

In yet another embodiment, the microbial cell further synthesizes a dehydrogenase from another microbial cell.

In even yet another embodiment, one or more housekeeping dehydrogenases are deactivated or engineered.

In a preferred embodiment, the microbial cell further synthesizes a dehydrogenase from another microbial cell and one or more housekeeping dehydrogenases are deactivated or engineered.

The dehydrogenase employed in this embodiment is according to the previous embodiments.

The co-factor may be NAD (P) H or NADH and the regeneration system is a NAD (P) H or NADH regeneration system. Preferably, the regeneration system is a NADH regeneration system.

The regeneration system is preferably co-expressed in the same microbial host which expresses the enzyme catalysing the oxidative transformation. In a more preferred embodiment, the same microbial host co-expresses also a dehydrogenase, as described in previous embodiments.

Cofactors are non-protein chemical compounds that play an essential role in many enzymes catalysed biochemical reactions. Cofactors act to transfer chemical groups between enzymes. Nicotinamide adenine dinucleotide (NAD+), and nicotinamide adenine dinucleotide phosphate (NADP+) and the reduced forms of said molecules (NADH and NADPH, respectively) are biological cofactors which play a central role in the metabolism of cells acting as electron transfer agents. The oxidized forms NAD+ and NADP+ act as electron acceptors, becoming reduced in the process. NADH and NADPH, in turn, can act as reducing agents, becoming oxidized in the process. Most enzymes that mediate oxidation or reduction reactions are dependent on cofactors such as NADPH or NADH. Cofactor regeneration systems are employed to ensure that the cofactor participating within a given bioprocess is not depleted and/or to reduce the total cost of the process.

In a preferred embodiment, the NADH regeneration system is a formate dehydrogenase regeneration system.

In another preferred embodiment, the NADH regeneration system is a formate dehydrogenase-based system, more preferably a cytosolic format dehydrogenase with no sensitivity towards oxygen.

In another preferred embodiment, the NADH recycling system is comprised of a metal-independent formate dehydrogenase active on NAD+ species and of bacterial or fungal origin.

Preferably, the metal-independent formate dehydrogenase, which is active on NAD+ species, is from Candida tropicalis or Mycobacterium vaccae FDH.

In a preferred embodiment, the formate is fed to the process, as defined in any of the previous embodiments, for regeneration of NADH consumed by the enzyme, which catalyzes the oxidative transformation of the methyl groups of 2,6-lutidine II to the respective hydroxymethyl groups of 2,6-bis(hydroxymethyl)pyridine I, the dehydrogenase, or both. Preferably, the formate is fed to the process, for regeneration of NADH consumed by the oxidoreductase, the dehydrogenase, or both.

In a preferred embodiment, the feeding rate of formate in the reaction medium is adjusted such that the concentration of formate does not exceed the value of 150 mM, preferably 100 mM, more preferably 50 mM.

In another preferred embodiment, the feeding rate of formate in the reaction medium is adjusted such that the concentration of formate does not fall below the value of 50 mM, preferably 25 mM, more preferably 5 mM, in the reaction medium.

In a more preferred embodiment, the feeding rate of formate in the reaction medium is adjusted such that the concentration of 2,6-lutidine II does not exceed the value of 150 mM and does not fall below the value of 50 mM, preferably 25 mM, more preferably 5 mM, in the reaction medium.

In another preferred embodiment, the feeding rate of formate in the reaction medium is adjusted such that the concentration of formate does not exceed the value of 100 mM and does not fall below the value of 50 mM, preferably 25 mM, more preferably 5 mM, in the reaction medium.

In another preferred embodiment, the feeding rate of formate in the reaction medium is adjusted such that the concentration of formate does not exceed the value of 50 mM and does not fall below the value of 50 mM, preferably 25 mM, more preferably 5 mM, in the reaction medium.

The invention will be further described on the basis of examples and figures, which show:

FIG. 1A. Time course of 2,6-bis(hydroxymethyl)pyridine formation from 2,6-lutidine catalyzed by a wild type monooxygenase XylMA from P. putida (squares) and a mutated XylMA harbouring the exchange of M to G at the position 116 (triangles)
B. Whole cell biotransformation of 1 g/L 2,6-lutidine II to 2,6-bis(hydroxymethyl)pyridine I by XMO WT and mutants harbouring substitutions at position M116. For simplicity, only the concentration of the target product I are plotted over the course of the biotransformations.

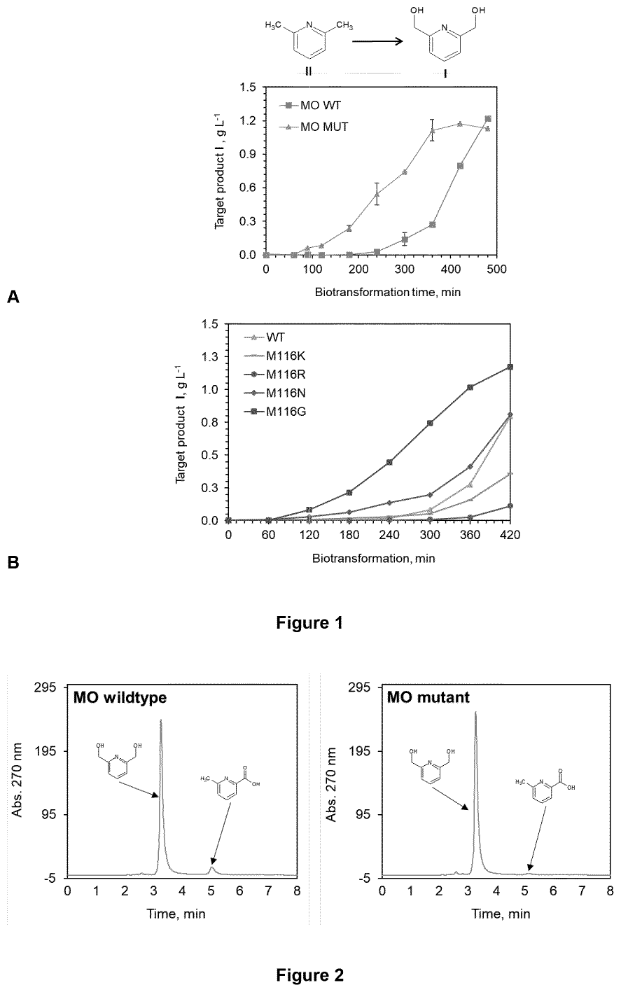

FIG. 2 HPLC-UV chromatograms arising from overnight lab-scale biotransformations using a wild type monooxygenase XylMA from P. putida (left) and a mutated XylMA harbouring the exchange of M to G at the position 116 (right). The peak area of the peak corresponding to the side product, 6-methyl-2-pyridinecarboxylic acid V.

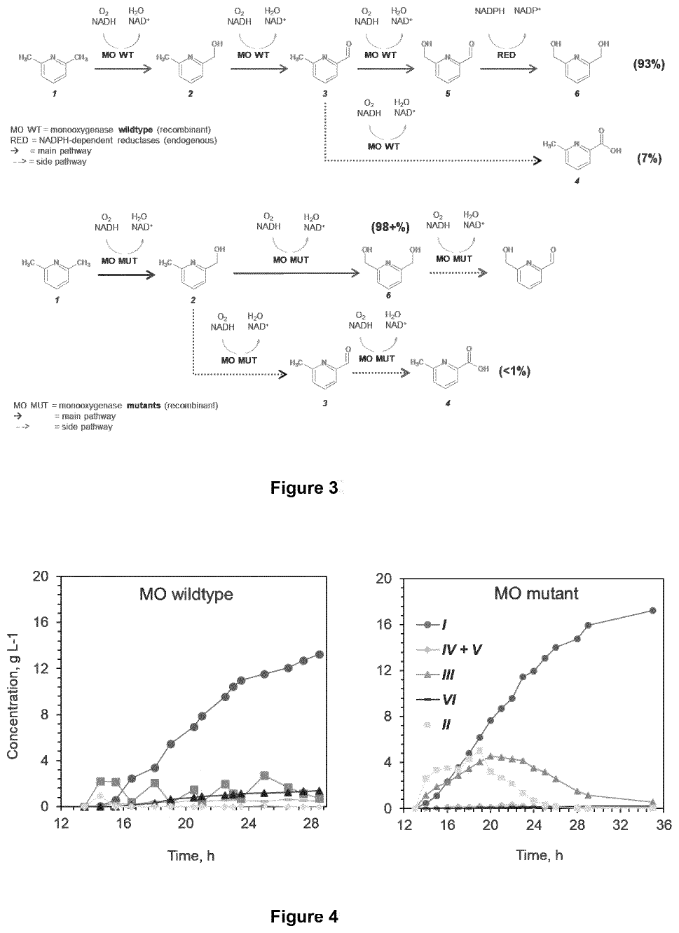

FIG. 3 Reaction schemes for conversion of 2,6-lutidine II to 2,6-bis(hydroxymethyl)pyridine I by XylMA wildtype (top) and XylMA mutant (bottom) which unlike the wildtype, efficiently catalyzes the reaction via direct hydroxylation of 6-methyl-2-hydroxypyridine III

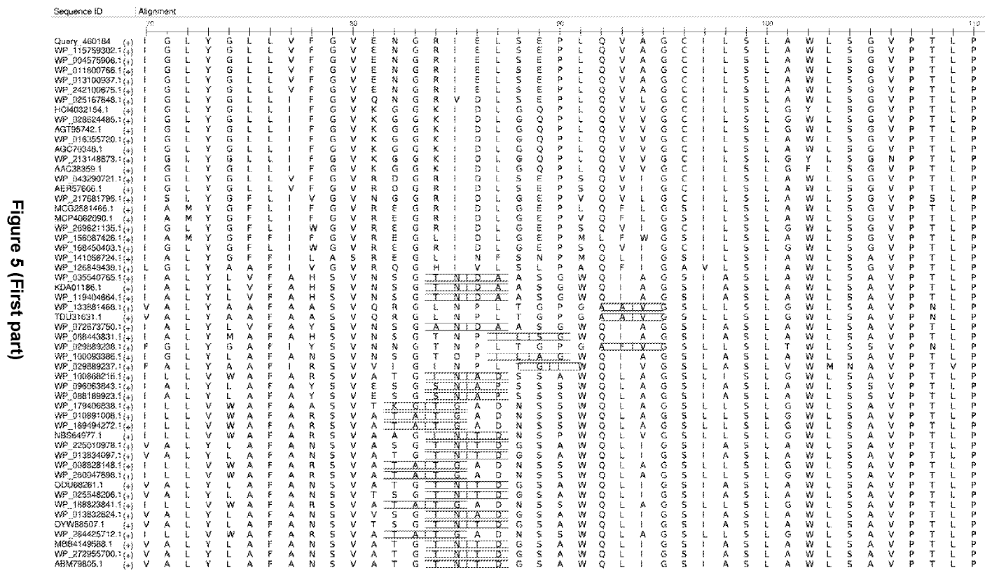

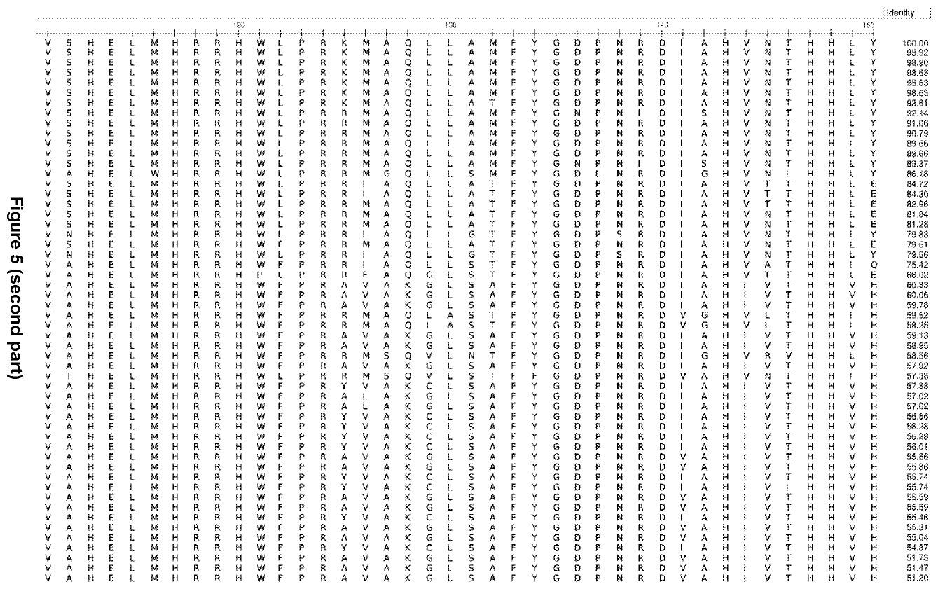

FIG. 4 Accumulation of intermediates and products in preparative synthesis of 2,6-bis(hydroxymethyl)pyridine I by XylMA wildtype (left) and XylMA mutant (right)

FIG. 5 Multiple sequence alignment showing that the highly conserved nature of the region around M116 in XylM from P. putida and sequences with homology >50% to the said protein.

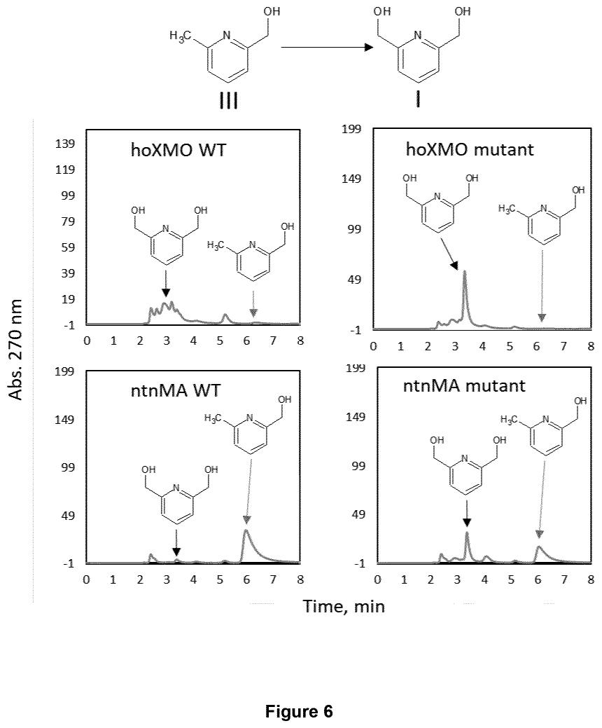

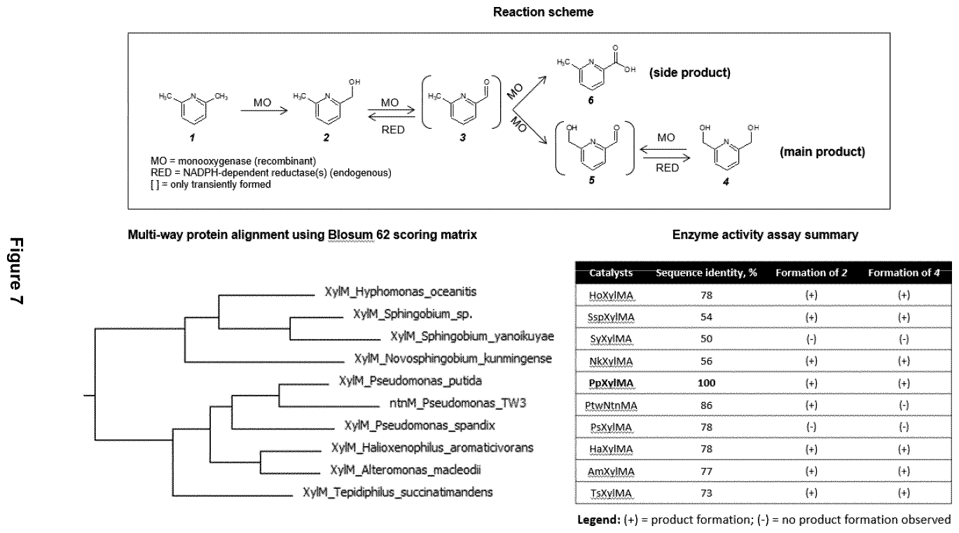

FIG. 6 HPLC chromatograms of overnight conversion of 0.5 g/L of compound III by two related proteins (GenBank: BBB44451.1 (hoXMO WT) and GenBank: AAC38359.1 (ntnMA WT) and two side directed mutagenesis mutants harbouring mutations at position 116.

FIG. 7 Phylogenetic relationship between different XylM and XylM-like enzymes and their enzymatic activity

### EXAMPLES

### Example 1: Genetic Manipulation of the Wild Type XylM Protein

Amino acid sequence of the WT
>sp|P21395|XYLM_PSEPU Xylene monooxygenase
subunit 1 OS = Pseudomonas putida OX = 303
GN = xylM PE = 3 SV = 1
MDTLRYYLIPVVTACGLIGFYYGGYWVWLGAATFPALMVLDVILPKDFS
ARKVSPFFADLTQYLQLPLMIGLYGLLVFGVENGRIELSEPLQVAGCIL
SLAWLSGVPTLPVSHELMHRRHWLPRKMAQLLAMFYGDPNRDIAHVNTH
HLYLDTPLDSDTPYRGQTIYSFVISATVGSVKDAIKIEAETLRRKGQSP
WNLSNKTYQYVALLLALPGLVSYLGGPALGLVTIASMIIAKGIVEGFNY
FQHYGLVRDLDQPILLHHAWNHMGTIVRPLGCEITNHINHHIDGYTRFY
ELRPEKEAPQMPSLFVCFLLGLIPPLWFALIAKPKLRDWDQRYATPGER
ELAMAANKKAGWPLWCESELGRVASI
https://www.uniprot.org/uniprot/P21395

Manipulation of the gene sequence SEQ ID NO: 1 with the purpose of introducing random mutations were carried out using standard techniques for random mutagenesis, i.e., error-prone PCR amplification of the xylM gene using mutagenic DNA polymerase. The resulting PCR product was cloned in a vector backbone, comprising pBR322 origin of replication, kan gene encoding kanamycin resistance protein and inducible Pₐₗₖₛ promoter for XylMA induction by dicyclopropyl ketone (DCPK) suitable for protein expression and transformed in the expression strain by electroporation. The presence of random mutations was confirmed by DNA sequencing. A library exceeding 50,000 unique variants was generated.

Screening for target activity was carried out using MALDI-MS. This protocol allowed the measurement of 384 samples (one 384-well microtiter plate) in roughly 20 minutes thereby allowing the screening of >20,000 clones from the library. The variants from wells in which the highest signal corresponding to the product of interest were re-screened in 96-well plate whereas product formation was quantified by HPLC-UV and the contained mutations in the gene sequence were identified by DNA sequencing.

### Example 2: Conversion of Lutidine by Recombinant E. coli Expressing XylMA Protein in Shake Flasks

The polynucleotide sequence of the xylM and xylA genes of Pseudomonas putida (Arthrobacter siderocapsulatus) encoding for multi-component xylene monooxygenase, XylMA, was cloned into plasmid comprising pBR322 origin of replication, kan gene encoding kanamycin resistance protein and inducible P_{alkS} promoter for XylMA induction by dicyclopropyl ketone (DCPK), transformed by electroporation into an E. coli BL21 host and plated on LB agar plates supplemented with kanamycin. After incubation over night at 37° C., a single colony was picked and propagated 37° C. in 4 mL LB growth medium om a shaker at 200 rpm for 12-14 h. On the following day, the overnight culture in LB was used to inoculate a main culture in minimal medium containing 4.5 g/L KH₂PO₄, 6.3 g/L Na₂HPO₄, 2.3 g/L (NH₄)₂SO₄; 1.9 g/L NH₄Cl; 1 g/L citric acid, 20 mg/L thiamine, 10 g/L glycerol, 55 mg/L CaCl₂, 240 mg/L MgSO₄, 1×trace elements (0.5 mg/L CaCl₂·2H₂O; 0.18 mg/L ZnSO₄·7H₂O, 0.1 mg/L MnSO₄·H₂O, 20.1 mg/L Na₂-EDTA, 16.7 mg/L FeCl₃·6H₂O, 0.16 mg/L CuSO₄·5H₂O), 50 mg/L kanamycin at pH 7 adjusted with NH₄OH. The starting optical density (OD600) of the 20 mL main culture in 100 mL shake flask was adjusted to 0.05 and the flask was incubated at 37° C., 200 rpm until OD of 0.6-0.8 was reached, then 0.025% DCPK was added and the culture was further incubated at 30° C., 200 rpm for another hour or until OD reached 1. At the target OD, various sub-growth-inhibitory concentrations of 2,6 lutidine II were added to the cells and the cultures were incubated further until complete substrate conversions was achieved and cell growth has stalled for at least 2 hours. The reaction progression was monitored and quantified using RP-HPLC equipped with a C18 column at 270 nm and a specific activity range of 0.3-0.6 g/gCDW/h were calculated for the individual reactions catalyzed by the whole cells. The results indicated formation of 1.25 g/L of hydroxylated products (93% 2,6-bis(hydroxymethyl)pyridine I; 5-7% 6-methyl-2-pyridinecarboxylic acid V).

FIG. 1 shows the lab-scale reaction progression and rate of target product accumulation when the reaction is catalysed by the wildtype monooxygenase (squares in FIG. 1a and triangles in FIG. 1b) versus different monooxygenase mutants. FIG. 1a shows results for the mutant harbouring amino acid exchange from M to G, wherein the required reaction product 2,6-bis(hydroxymethyl)pyridine starts to accumulate earlier and also reaches maximum concentration faster (approximately 2 hours earlier). FIG. 1b shows the comparison between individual preferred mutated monooxygenases, wherein the reaction rates and final yields are higher for the mutant having the amino acid sequence SEQ ID NO 2 and SEQ ID NO 3. Although, the other two mutants having the amino acid sequence SEQ ID NO 4 and SEQ ID NO 5 have a lower yield and lower reaction rates, the amount of toxic side products is still significantly reduced in comparison to the wild type, hence these mutants are still preferred to the WT.

FIG. 2 demonstrates differences in final product profile, i.e., HPLC-UV chromatograms generated by the wildtype monooxygenase (left) and mutant monooxygenase harbouring amino acid exchange M to G (thus having sequence SEQ ID NO: 2) (right). The chromatograms were obtained from overnight shake flask biotransformations as described above. The peak area of the peak corresponding to the side product, 6-methyl-2-pyridinecarboxylic acid V is far less significant in the product profile obtained with the mutated enzyme indicating superior catalysis, improved efficiency and lower toxicity.

FIG. 3 illustrates these differences in reaction schemes for conversion of 2,6-lutidine II to 2,6-bis(hydroxymethyl)pyridine I by XylMA wildtype (top) and XylMA mutant (SEQ ID NO: 2; bottom) which unlike the wildtype, efficiently catalyzes the reaction via direct hydroxylation of 6-methyl-2-hydroxypyridine III.

### Example 3: Conversion of 2,6-Lutidine by Recombinant E. coli Expressing XylMA Protein in a Bioreactor

The microbial strain, media and growth conditions up to inoculation of the main culture are identical to example 1. However, in this example, the main culture is prepared in a bioreactor, where parameters such as temperature, pH, dissolved oxygen tension, mixing and glucose availability can be controlled, allowing for fed batch fermentations. Fluctuations in pH are maintained by appropriate addition of ammonium hydroxide or sulfuric acid controlled by a pH-stat. For the batch phase of the fermentation, 1 L growth media (as in example 1) was inoculated at a starting OD600 of 0.025 and cells were grown at 30° C. for 12-13 h or until they completely consumed the initially provided carbon source (e.g. glucose or glycerol) which is indicated by a sharp jump in dissolved oxygen in the bioreactor. At this stage, the fed-batch phase of the fermentation was initiated by the addition of appropriate glucose at an appropriate feed rate from a 500 g/L glucose stock supplemented with 1× trace elements, 1× kanamycin and 240 mg/L MgSO₄ such that a growth rate of 0.31 h⁻¹ was maintained until OD600 reached 35 when 0.05% DCPK were added. One hour post induction with DCPK, 2,6-Lutidine II was added to the bioreactor (feed rate: 0.1 mL/L of broth/min) and the reaction was let to proceed for 14-18 h. A second addition of 2,6-lutidine II can be made once the initially supplied amount is converted to 2,6-bis(hydroxymethyl)pyridine I and the reaction is let to proceed until conversion is completed or as long growth rate of the cells higher than 0.025 h⁻¹ is maintained. Up to 20 g/L total product (90% 2,6-bis(hydroxymethyl)pyridine I; 10% 6-methyl-2-pyridinecarboxylic acid V) could be produced within 20 h biotransformation.

FIG. 4 shows accumulation of intermediates and products in preparative synthesis of 2,6-bis(hydroxymethyl)pyridine I by XylMA wildtype (left) and XylMA mutant (right). The formation of the main product, side products and intermediates formed in the course of the biotransformation of 2,6-lutidine II catalyzed by wildtype monooxygenase and monooxygenase mutant M116G are separately shown. The preparative reaction catalyzed with mutant M116G yielded ˜30% more product under non-optimized bioprocess conditions. Optimization of bioprocess conditions and the use of mutant M116G is expected to bring >50%. In addition, the average rate of target product formation increased from 0.8 g/L/h (monooxygenase wildtype) to 1 g/L/h (monooxygenase M116G mutant) and compounds II and III are converted by the mutant simultaneously, not hierarchically. Lastly, significantly reduced side-product formation is realized with the mutant (from ˜5% (wildtype) to <1% (mutant M116G)).

### Example 4: Conversion of 2,6-Lutidine by Recombinant E. coli Expressing XylMA-Like Proteins

The introduction of exchanges at the functional equivalent of M116 in P. putida has similar effect on the biotransformation efficiency. We showed this with the xylM-like gene from H. aromaticivorans. The H. aromaticivorans mutant having mutations at position equivalent to M116 showed reaction improved rates, higher yields and simultaneous (not hierarchical) conversion of compounds of formula II and formula III.

FIG. 5 shows multiple sequence alignment indicating that the region around M116 in XylM from P. putida (highlighted in figure with a box) is highly conserved among related sequences with sequences having more than 50% identity to XylM from P. putida. In order to show that the replacement of methionine at position 116 or equivalent causes the same effect in related enzymes, two mutated enzymes were prepared by targeted mutagenesis and expressed in E. coli host with reduced aromatic aldehyde reduction capacity (E. coli RARE). HPLC chromatograms of overnight, i.e., 1020 minute reaction, conversions of 0.5 g/L of compound III by two related proteins (GenBank: BBB44451.1 (hoXMO WT) and GenBank: AAC38359.1 (ntnMO WT) and two side directed mutagenesis mutants harboring mutations at position 116 are shown. The mentioned E. coli host allows that the desired product I can be only formed via direct hydroxylation of the free methyl group of compound III catalysed by the tested enzymes (two WT and two mutants). In both cases, the amino acid exchange in the mutant enzymes significantly improves the formation of the desired product, as was the case with mutated XylM from P. putida. Only the peaks corresponding to the substrate (grey arrow) and the desired product (black arrow) are indicated on the figure. The additional peaks correspond to overoxidation products (aldehydes, acids) are not explicitly highlighted. Both mutants show an increase in the desired products, while the amount of the reactant is significantly lower.

FIGS. 6 and 7 show results for various XylM-like enzymes, having at least 50% homology to the P. putida enzyme. As shown in FIG. 6 HPLC chromatograms of overnight conversion of 0.5 g/L of compound III by two related proteins (GenBank: BBB44451.1 (hoXMO WT) and GenBank: AAC38359.1 (ntnMA WT) and two side directed mutagenesis mutants harbouring mutations at position 116 were shown to have improved product yield and less side products. Similarly, FIG. 7 shows data for more XylM-like enzymes, namely from the following species Hyphomonas oceanitis, Spingobium sp., Novosfingobium kunmingense, Pseudomonas TW3, Halioxenophilus aromaticivorans, Alteromonas macleodii and Tepidiphilus succinatimandens all show enzyme activity similar to XylM of P. putida. Since the homology of amino acid sequences are at least 50%, these results indicate that this is sufficient homology to retain the enzyme activity and show improved yields with the above-described mutations.

These results confirm the importance and the effect of amino acid exchange at position 116 or equivalent, where methionine or tryptophan is present in the wild type enzymes.

1. An enzyme having a sequence SEQ ID NO: 1 or at least 50% homology on the amino acid level to the said sequence, said homology ensuring enzymatic activity of the said enzyme, said protein having a mutation at position 116 or an equivalent position, wherein the mutation is a replacement of methionine (M) or tryptophan (W) by a different amino acid.

2. The enzyme according to claim 1, wherein the M or W at position 116 or equivalent is replaced with an amino acid selected in the group consisting of G, N, R, or K, preferably with G.

3. The enzyme according to claim 1 having further mutations and/or deletions.

4. The enzyme according to claim 1, wherein the enzyme is a:
XylMA enzyme of Pseudomonas putida, or
XylMA-like enzyme of
Alteromonas macleodii, or
Tepidiphilus succinatimandens, or
Novosphingobium kunmingense, or
Hyphomonas oceanitis, or
Sphingobium sp. 32-64-5 or
Halioxenophilus aromaticivorans or
a XylMA-like enzyme with more than 50% sequence identity to SEQ ID NO: 1 on the amino acid level.

5. A nucleic acid encoding the enzyme according claim 1.

6. An expression vector comprising the nucleic acid according to claim 5.

7. A host cell with the nucleic acid and/or the expression vector expressing the enzyme according to claim 1.

8. The host cell according to claim 7, wherein the host cell is a microbial cell, preferably a bacterial cell.

9. The host cell according to claim 8, wherein the host cell is a cell of Escherichia coli, Corynebacterium glutamicum, Bacillus subtilis, Pseudomonas putida, Rhodobacter sphaeroides, Streptomyces spp, Propionibacterium shermanii, Ketogulonigenium vulgare, Acinetobacter baylyi, Halomonas bluephagenesis, most preferably an E. coli cell.

10. Use of the enzyme, the nucleic acid, and/or the host cell according to claim 1, claim 5, or claim 7 in a process for the transformation of 2,6-lutidine II to 2,6-bis(hydroxymethyl)pyridine I.

11. A process for the transformation of 2,6-lutidine II to 2,6-bis(hydroxymethyl)pyridine I, 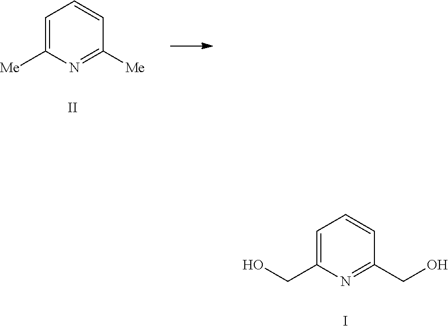
wherein the transformation is performed in the presence of enzymes, characterized in that the enzyme or the host cell according to claim 1 or claim 7 is used.

12. The process according to claim 11 wherein the feeding rate of 2,6-lutidine II in the reaction medium is adjusted such that the concentration of 2,6-lutidine II does not exceed the value of 1 g/L, preferably 0.1 g/L, and more preferably 0.02 g/L in a reaction medium, and wherein the feeding rate of 2,6-lutidine II in the reaction medium is adjusted such that the concentration of 2,6-lutidine II does not fall below the value of 10 mg/L, preferably 0.1 mg/L, more preferably 0.01 mg/L.

13. A process according to claim 8 or 9, wherein a dehydrogenase is used, wherein the dehydrogenase is preferably co-expressed in the microbial host.

14. A process according to claim 13, wherein the dehydrogenase is NADH dependent, NADP dependent, NADPH dependent or GDH dependent, wherein the dehydrogenase is preferably selected from the list of the AKR from Kluyveromyces lactis, XylB from Acinetobacter baylyi ADP1, and AFPDH from Candida maris.

15. A process according to claim 13, wherein a NADH regeneration system, a NADP regeneration system, a NADPH regeneration system or a GDH regeneration system is co-expressed in the microbial host, wherein the NADH regeneration system is preferably a formate dehydrogenase-based system, wherein the NADH regeneration system is preferably comprised of a metal-independent formate dehydrogenase active on NAD+ species and of bacterial or fungal origin.

16. A process according to claim 15, wherein the feeding rate of formate is such that the concentration of formate in the reaction medium does not exceed the value of 150 mM, preferably 100 mM, more preferably 50 mM, and wherein the feeding rate of formate is such that the concentration of formate does not fall below the value of 50 mM, preferably 25 mM, more preferably 5 mM, in the reaction medium.

17. Use of product of the process according to claim 11 in preparation of other compounds, diagnostic complexes, most preferably 2-[3,9-bis[1-carboxylato-4-(2,3-dihydroxypropylamino)-4-oxobutyl]-3,6,9,15-tetraza bicyclo[9.3.1]pentadeca-1 (15),11,13-trien-6-yl]-5-(2,3-dihydroxypropylamino)-5-oxopentanoate;gadolinium(3+), 2,6-Bis(chloromethyl)pyridine, 2,6-Bis(bromomethyl)pyridine, 2,6-Bis(mesyloxymethyl)pyridine and 2,6-Bis(tosyloxymethyl)pyridine.

18. A process for preparation of pyridine-based tetra-aza heterocycles, preferably 3,6,9,15-tetraazabicyclo[9.3.1]pentadeca-1(15),11,13-triene, i.e., pyclen, comprising the method and/or products of the method according to claim 11.

19. A process for the preparation of various diagnostic complexes or other compounds comprising the method and/or products of the method according to claim 11.

20. The process according to claim 19, wherein the process is preparation of 2-[3,9-bis[1-carboxylato-4-(2,3-dihydroxypropylamino)-4-oxobutyl]-3,6,9,15-tetraza bicyclo[9.3.1]pentadeca-1(15),11,13-trien-6-yl]-5-(2,3-dihydroxypropylamino)-5-oxopentanoate;gadolinium (3+), i.e., gadopiclenol.

21. A process according to claim 10, wherein a dehydrogenase is used, wherein the dehydrogenase is preferably co-expressed in the microbial host.

22. A process according to claim 11, wherein a dehydrogenase is used, wherein the dehydrogenase is preferably co-expressed in the microbial host.

23. A process according to claim 12, wherein a dehydrogenase is used, wherein the dehydrogenase is preferably co-expressed in the microbial host.


SEQUENCE LISTING (6 sequences)

SEQ ID NO: 1 - protein, 369 aa, Pseudomonas putida
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLPRKMAQLLAMFYGDPNRDIAHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 2 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELGHRRHWLPRKMAQLLAMFYGDPNRDIAHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 3 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELNHRRHWLPRKMAQLLAMFYGDPNRDIAHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 4 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELKHRRHWLPRKMAQLLAMFYGDPNRDIAHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 5 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELRHRRHWLPRKMAQLLAMFYGDPNRDIAHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 6 - protein, 363 aa, Halioxenophilus aromaticivorans
MDTIRYYLIPLVSACGALGFYYGGDWVWLGAATFPSLMILDVLLPRDYEERKVSPFFADLTQYLQLPLMIAMYGFLIFGVREGRIDLGEPVQFLGSILSLAWLSGVPTLPVSHELMHRRHWLPRRMAQLLATFYGDPNRDIAHVNTHHLELDTPLDSDTPFRGQTMYSFVVSATVGSVMDAAKIEAETLRRKGKSPWHLSNKMYQYVMLLIALPGVVTYFGGAESGLVTIISMLIAKAIVEGFNYFQHYGLVREIGHPILLHHAWNHMGMIVRPLGCEITNHINHHLDGYTRFYYLHPEKEAPQMPSLFLCFLLGLVPPLWENLVAKPKLKDWDLQYATPGERKLAMEANKNAGWPQWIPEAA